(19) United States
(12) Patent Application Publication
(10) Pub. No.: US 2002/0169426 A1
(43) Pub. Date: Nov. 14, 2002

(54) Injection needle

(76) Inventor: Hiroshi Takagi, Yokohama-shi (JP)

Correspondence Address: KUBOVCIK & KUBOVCIK, SUITE 710, 900 17TH STREET NW, WASHINGTON, DC, 20006

(21) Appl. No.: 10/134,393

(22) Filed: Apr. 30, 2002

(30) Foreign Application Priority Data
May 11, 2001 (JP) 2001-142365

(51) Int. Cl. A61M 5/32

(52) U.S. Cl. 604/272

(57) ABSTRACT

An object of the present invention is to provide an injection needle which has a low insertion resistance, which can inhibit an occurrence of a force pushing the needle outward, and which can prevent the coring. Moreover, another object of the present invention is to provide an injection needle which ensures a certain degree of flow rate when fluid flows in and out and which has a low inflow-outflow resistance. For these objects, there is provided an injection needle comprising a rigid hollow needle and a needle hub at the root end of the hollow needle, wherein the hollow needle comprises a flat surface at its tip end and a sloped surface from the flat surface to the needle hub.

### BACKGROUND OF THE INVENTION

1. Field of the Invention

The present invention relates to an injection needle through which fluid is supplied.

2. Description of the Related Art

It has been known that a metal needle with a tip end is used for supplying drugs and taking blood samples. When using metal needles, however, medical workers may meet with so-called needle prick accidents. Moreover, for patients who are frequently given injections of drugs or taken blood samples, since metal needles are pricked into their skin repeatedly, their injection sites sometimes get rough or they can sometimes be inflicted with mental pain.

As means for solving the above problems, there has been known a method in which a catheter is inserted into a patient, the catheter is connected to a tube, and the tube is connected to a three-way stopcock. And drugs are supplied to the patient through a metal needle inserted into the three-way stopcock. This method can just solve the above problems since it can decrease the number of times of pricking metal needles into the human.

However, the above-described method, in which a three-way stopcock is used, also has a problem such that the risk of bacterial infections is very high. In addition, since metal needles are used, medical workers may still meet with needle prick accidents.

Thus, as means for solving the problems attendant to the use of the three-way stopcock, there has been proposed a blood collecting system by a drug supplying apparatus, which uses a resin needle instead of a metal needle and a stopcock made of compressed rubber with a slit instead of a three-way stopcock. In this system, the resin needle is inserted into the slit of the stopcock when supplying drugs. Also in this system, after supplying the drugs, the needle is drawn out and the stopcock is closed by the action of the compressed rubber. Thus, the system functions as a closed system, making it possible to reduce the risk of patients being exposed to bacterial infections.

As shown in FIG. 4, the tip of the plastic needle conventionally used in this system has a large tip diameter and the flow rate is ensured. However, this has problems that the tip opening is so flat and thick that its insertion resistance becomes large, and that the needle slips off by the force pushing the needle outward. Furthermore, the plastic needle in this system has a problem of coring that a part of the compressed rubber or the like is scooped at the time of inserting, and that the scooped part enters the flow path or the blood vessels.

As shown in FIG. 5., there is also a plastic needle which has a conical portion at its tip with its end formed a flat surface 2b. The construction of the needle is such that in the conical portion is provided an opening 5 through which fluid flows in and out. This type of needle has problems that, though the resistance at the time of inserting is decreased, resistance to the inflow and outflow of the fluid occurs since the flow direction of the fluid is perpendicular to the axial direction, and that the flow rate is decreased since the opening 5 is narrowed.

### SUMMARY OF THE INVENTION

Accordingly, the object of the present invention is to solve the above-described problems.

Specifically, an object of the present invention is to provide an injection needle which has a low insertion resistance, which can inhibit an occurrence of a force pushing the needle outward, and which can prevent the coring.

Another object of the present invention is to provide an injection needle which ensures a certain degree of flow rate when fluid flows in and out and which has a low inflow-outflow resistance.

In order to accomplish the above objects, the present invention is as follows.

First, the present invention relates to an injection needle comprising a rigid hollow needle and a needle hub at the root end of the hollow needle, wherein the hollow needle comprises a flat surface at its tip end and a sloped surface from the flat surface to the needle hub.

As materials for the hollow needle, not only the materials limited as described later but also various resins can be used. The hollow needle is preferably transparent. This is because fluid can be visually observed effectively.

This type of injection needle can be manufactured by injection molding or extrusion molding followed by cutting.

Preferably the edge portions of its flat surface and sloped surface are chamfered so as to decrease the insertion resistance.

The injection needle is generally used with its needle hub fitted to a syringe to contain the fluid.

However, the needle hub is not necessarily fitted to a syringe, but may be fitted to a tube through which the fluid is carried.

According to the above invention, since the flat surface is formed at the tip end of the hollow needle, the hollow needle cannot easily prick human's body, whereby occurrence of a needle prick accident can be inhibited. Further, the injection needle according to the present invention has a low insertion resistance, inhibits the occurrence of a force to push the needle outward, and can prevent the occurrence of coring since the tip end enters the stopcock just like a wedge when inserting. Further, according to the present invention, the opening at the tip end of the hollow needle is kept large and thus an injection needle, which ensures a certain degree of flow rate when the fluid flows in and out with a low inflow-outflow resistance, can be provided.

The present invention may be an injection needle, which is a limited form of the above invention, wherein the hollow needle is tapered so as to increase its diameter toward the needle hub.

In the invention, the injection needle is allowed to have so rigid a structure that distortion or crush does not occur, since the hollow needle is tapered so as to increase its diameter toward the needle hub. The tapered injection needle is effective particularly when using resins as a material for the needle.

The present invention may be an injection needle, which is a limited form of the above invention, wherein a thermoplastic resin with a bending strength (measured according to ASTM D790) of 80 MPa or more is used as a material for forming the rigid hollow needle.

The examples of the thermoplastic resins with a bending strength (measured according to ASTM D790) of 80 MPa or more are polyesters such as crystalline polyethylene naphthalate and noncrystalline polyethylene terephthalate, and polyolefins such as cyclicolefin copolymer and noncrystalline cycloolefin copolymer, etc.

The injection needle of the invention uses a thermoplastic resin with a bending strength (measured according to ASTM D790) of 80 MPa or more, whereby distortion or crush of the hollow needle at the time of insertion can be prevented. In addition to the above condition, if the hollow needle is tapered as limited above, distortion or crush can be prevented more effectively.

The present invention may be an injection needle, which is a limited form of the above invention, wherein the tip of the hollow needle has a virtual inner diameter of 1.0 mm to 1.2 mm and a virtual outer diameter of 1.4 mm to 1.8 mm.

When the tip of the hollow needle has a virtual inner diameter of 1.0 mm to 1.2 mm, the flow rate per unit time can be increased. And when the tip of the hollow needle has a virtual outer diameter of 1.4 mm to 1.8 mm to make its thickness thin, the insertion resistance can be considerably decreased.

Due to the sloped surface formed at the tip of the hollow needle, the hollow needle actually does not have a tubular form. Thus the terms “virtual inner diameter” and “virtual outer diameter” herein used mean the inside diameter and the outside diameter, respectively, assuming that the tip of the hollow needle may have a tubular form.

The present invention may be an injection needle, which is a limited form of the above invention, wherein an angle between the sloped surface and the axis of the needle is not less than 25 degrees and not more than 35 degrees.

When the angle is within the above range, the insertion resistance can be decreased and the aspiration of the fluid into a syringe can be performed reliably.

If the angle is less than 25 degrees, there occurs a problem that the opening area of the hollow needle becomes so large that the air may flow into a syringe when aspirating the fluid into the syringe. If the angle is more than 35 degrees, there occurs a problem that the insertion resistance becomes large and that coring is likely to occur.

### BRIEF DESCRIPTION OF THE DRAWINGS

FIG. 1 shows a perspective view of an injection needle according to an embodiment of the present invention.

FIG. 2 shows enlarged views of the tip end portion of the embodiment of the present invention.

FIG. 3 shows a perspective view illustrating the state in which the injection needle according to the embodiment of the present invention accesses an injection portion.

FIG. 4 shows a perspective view of an injection needle of a prior art.

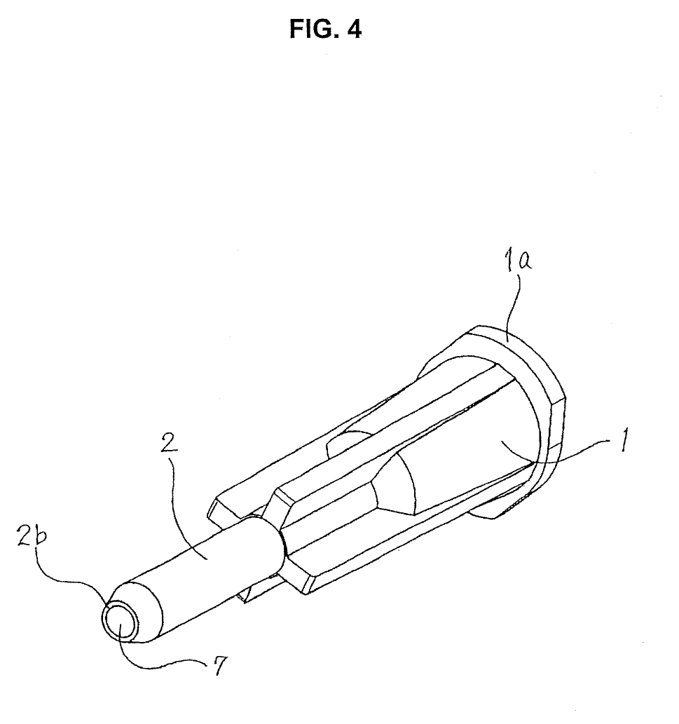

FIG. 5 shows a perspective view of an injection needle of another prior art.

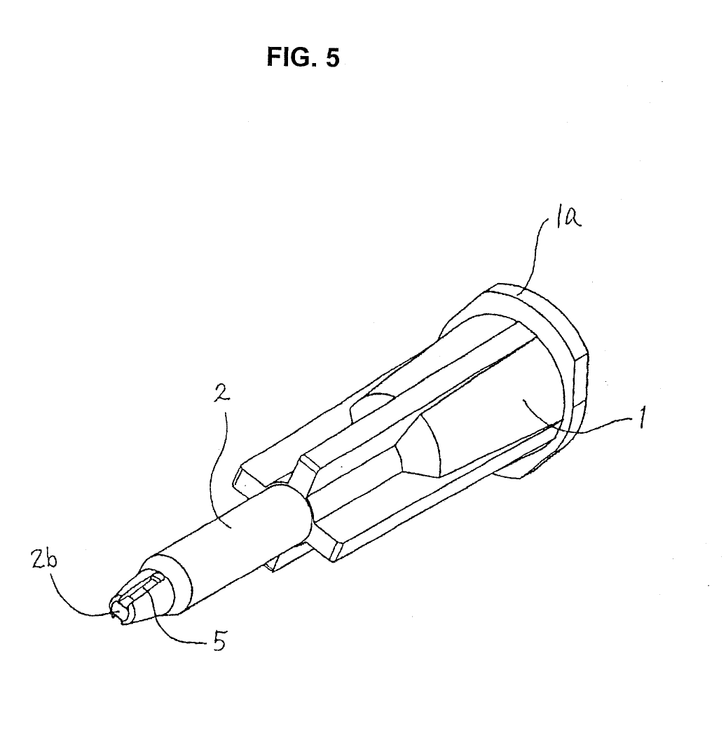

### DETAILED DESCRIPTION OF THE PREFERRED EMBODIMENTS

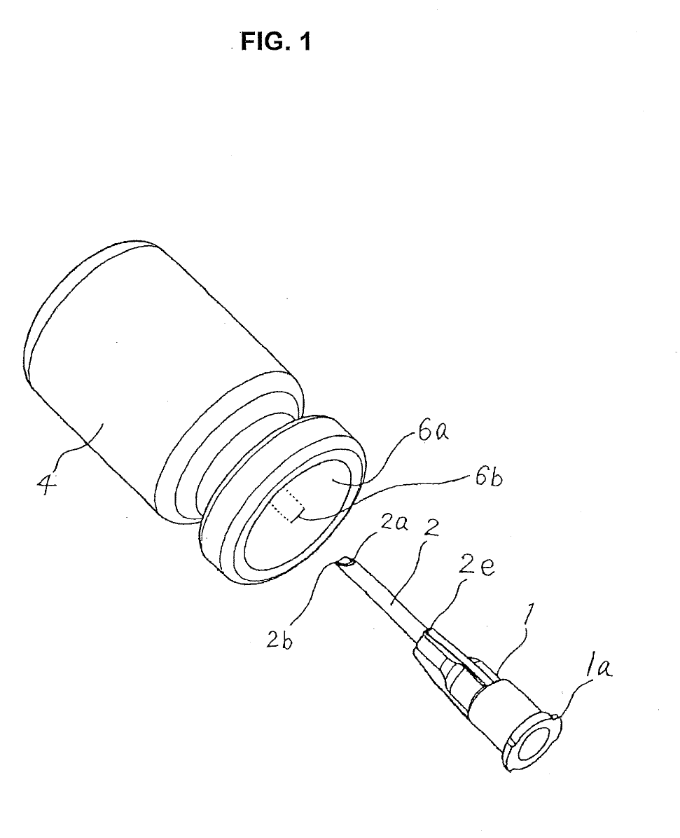

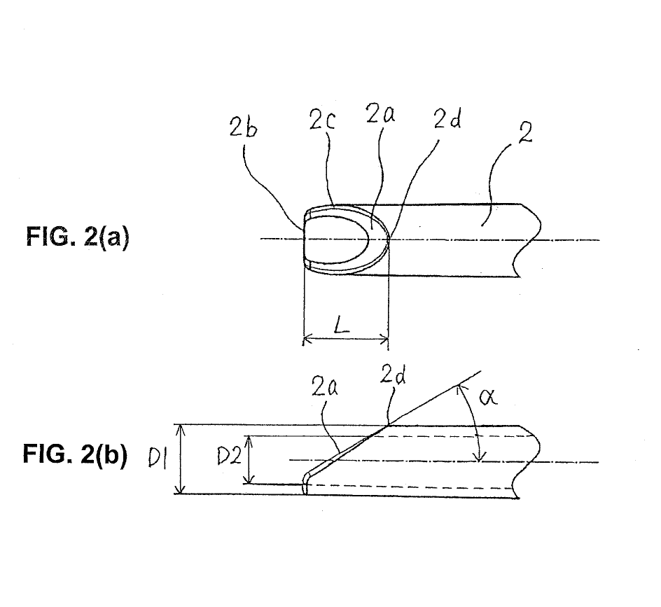

FIG. 1 shows a perspective view of an injection needle according to an embodiment of the present invention, and FIGS. 2(a) and (b) show enlarged views of the tip end portion of the embodiment of the present invention. The injection needle in the embodiment consists of a needle hub 1 and a hollow needle 2. At the rear end of the needle hub 1 is provided a guard portion 1a. In the vicinity of the guard portion 1a is attached a syringe, a tube or the like supplying fluid (not shown in the figure). At the tip of the hollow needle 2 are formed a sloped surface 2a and a flat surface 2b, and at their edges is provided a chamfered portion 2c. The chamfered portion 2c can reduce the resistance generated when inserting the injection needle into a slit 6b.

The hollow needle 2 can be inserted into the slit 6b on a compressed rubber 6a provided for a vial 4. After inserting, various types of fluid are drawn in and out through the hollow needle 2. The slit 6 is usually in the closed state, and thus the smaller the area of the flat surface 2b is, the lower the resistance becomes when inserting. Further, the outer diameter of the hollow needle 2 is as small as 1.7 to 1.9 mm, and, in this respect, the insertion resistance of the hollow needle can be decreased.

The virtual outer diameter D1 of the tip portion is 1.7 mm and the diameter of the root portion 2e 1.9 mm. Thereby the hollow needle 2 shows a tapered appearance. The material used for the hollow needle 2 is noncrystalline polyethylene terephthalate and its bending strength (measured according to ASTM D790) is 85 MPa. This allows the injection needle to have so rigid a structure that distortion or crush does not occur in it. The virtual inner diameter D2 of the tip of the hollow needle 2 is 1.15 mm. The longitudinal distance L from the tip of the hollow needle 2 to the rear end of the sloped surface 2d is about 2.1 mm. The angle α between the sloped surface 2d and the axis of the needle is about 30 degrees.

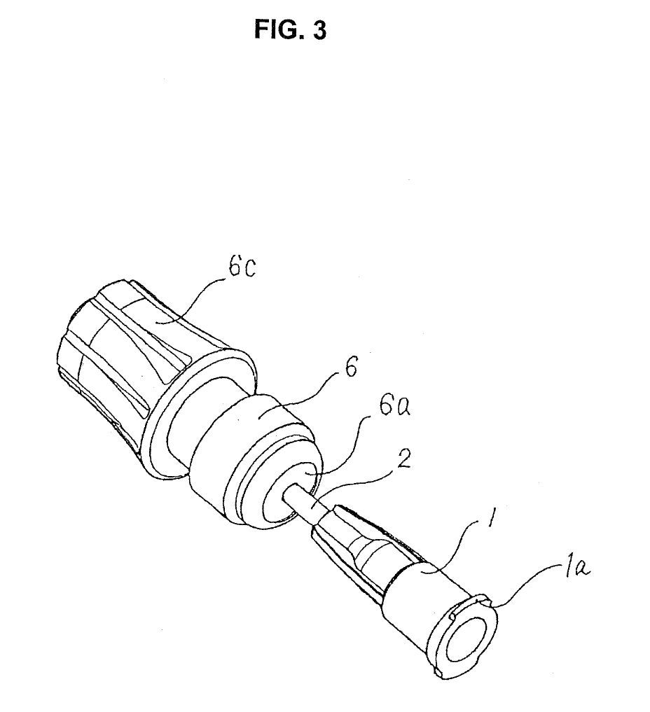

FIG. 3 shows a perspective view illustrating the state in which the injection needle according to the embodiment of the present invention accesses an injection portion 6. At the injection portion 6 is fitted a compressed rubber 6a provided with a slit into which the hollow needle 2 is inserted. On the other hand, at the far side from the compressed rubber 6a is provided a connecting portion 6c, which can be connected to other lines such as tubes.

Industrial Applicability

According to the present invention, it becomes possible to provide an injection needle which has a low insertion resistance, which can inhibit an occurrence of a force pushing the needle outward, and which can prevent the coring.

Moreover, it also becomes possible to provide an injection needle which ensures a certain degree of flow rate when fluid flows in and out and which has a low inflow-outflow resistance.

What is claimed is:

1. An injection needle comprising:
a rigid hollow needle; and
a needle hub at the root end of the hollow needle;
wherein the hollow needle comprises a flat surface at its tip end and a sloped surface from the flat surface to the needle hub.

2. The injection needle according to claim 1, wherein the hollow needle is tapered so as to increase its diameter toward the needle hub.

3. The injection needle according to claim 1, wherein a thermoplastic resin with a bending strength (measured according to ASTM D790) of 80 MPa or more is used as a material for forming the rigid hollow needle.

4. The injection needle according to claim 2, wherein a thermoplastic resin with a bending strength (measured according to ASTM D790) of 80 MPa or more is used as a material for forming the rigid hollow needle.

5. The injection needle according to claim 1, wherein the tip of the hollow needle has a virtual inner diameter of 1.0 mm to 1.2 mm and a virtual outer diameter of 1.4 mm to 1.8 mm.

6. The injection needle according to claim 2, wherein the tip of the hollow needle has a virtual inner diameter of 1.0 mm to 1.2 mm and a virtual outer diameter of 1.4 mm to 1.8 mm.

7. The injection needle according to claim 3, wherein the tip of the hollow needle has a virtual inner diameter of 1.0 mm to 1.2 mm and a virtual outer diameter of 1.4 mm to 1.8 mm.

8. The injection needle according to claim 4, wherein the tip of the hollow needle has a virtual inner diameter of 1.0 to 1.2 mm and a virtual outer diameter of 1.4 mm to 1.8 mm.

9. The injection needle according to claim 1, wherein an angle between the sloped surface and the axis of the needle is not less than 25 degrees and not more than 35 degrees.

10. The injection needle according to claim 2, wherein an angle between the sloped surface and the axis of the needle is not less than 25 degrees and not more than 35 degrees.

11. The injection needle according to claim 3, wherein an angle between the sloped surface and the axis of the needle is not less than 25 degrees and not more than 35 degrees.

12. The injection needle according to claim 4, wherein an angle between the sloped surface and the axis of the needle is not less than 25 degrees and not more than 35 degrees.

13. The injection needle according to claim 5, wherein an angle between the sloped surface and the axis of the needle is not less than 25 degrees and not more than 35 degrees.

14. The injection needle according to claim 6, wherein an angle between the sloped surface and the axis of the needle is not less than 25 degrees and not more than 35 degrees.

15. The injection needle according to claim 7, wherein an angle between the sloped surface and the axis of the needle is not less than 25 degrees and not more than 35 degrees.

16. The injection needle according to claim 8, wherein an angle between the sloped surface and the axis of the needle is not less than 25 degrees and not more than 35 degrees.
